(19) United States
(12) Patent Application Publication
(10) Pub. No.: US 2004/0110230 A1
(43) Pub. Date: Jun. 10, 2004

(54) Method for determining concentrations of analytes

(76) Inventors: Rainer Feldbrugge, Munster (DE); Andreas Gorschluter, Hamm (DE); Meinhard Knoll, Steinfurt (DE)

Correspondence Address: Barnes & Thornburg, 11 South Meridian Street, Indianapolis, IN, 46204, US

(21) Appl. No.: 10/450,321

(22) PCT Filed: Dec. 12, 2001
(86) PCT No.: PCT/EP01/14608

(30) Foreign Application Priority Data
Dec. 14, 2000 (DE) 100 62 173.2

(51) Int. Cl. G01N 33/53; G01N 33/50

(52) U.S. Cl. 435/7.1; 205/777.5

(57) ABSTRACT

The present invention relates to a method and a device for detecting and/or determining the concentration of analytes in a fluid to be analysed, for example a liquid. Methods and devices of this type are required in the field of analytical chemistry.

The present invention relates to a method and a device for detecting and/or determining the concentration of analytes in a fluid to be analysed, for example a liquid. Methods and devices of this type are required in the field of analytical chemistry.

From prior art, for example DE 197 51 706 A1 is known a method in which microparticles, the electrical properties of which are different from those of the measurement solution surrounding them, are used to detect analytes. The microparticles here bind specifically to the analyte, or competitively in relation to the analyte, on a substrate serving as a support. The analytes are then detected by the alteration of an electrical field generated by electrodes, which are caused by microparticles bound to them or instead of them by microparticles bound to the substrate.

Consequently, according to prior art, for the current between a measurement electrode and a counter-electrode in an electrolytic solution, an alteration is predicted if a particle having different dielectric properties from those of the electrolytic solution is located in the immediate vicinity of the electrode. This current alteration is caused by the alteration of the electrical field, induced by the particle, before the electrode, a measurement taking place without any conversion of matter. The size of the measurement signal depends on how many particles are located in the immediate vicinity of the electrode. However the alteration of the current does not show here any linear dependence on the number of the particles in the immediate vicinity of the electrode.

In the case of this method according to the prior art, the alteration of the electrical field depends on the concentration of the electrolyte with increasing distance from an electrode located in the electrolyte. The particle-induced alterations of the electrical field are also dependent on the electrolyte concentration. With identical concentration of analytes, different signals can also be produced, since the analyte to be determined is always present in an electrolyte matrix, the composition of which is often not known. For calibration, therefore, cleaning of the sample and of the particle suspension is necessary.

What is furthermore disadvantageous about the method according to the prior art, is that the measurement signal depends on the electrolyte concentration and thus is temperature-dependent. This also makes calibration difficult. The analysis is furthermore impaired by possible drift of the measuring current.

Furthermore in the method according to the prior art it is disadvantageous that only the number of bound particles on the electrodes can be established; however no statement can be made about the number of bonds with which a particle is held on an electrode.

The object of the present invention, therefore, is to make available a method and a device for detecting and/or determining the concentration of analytes in a fluid to be analysed, with great accuracy and specificity.

This object is accomplished by the method according to claim 1 and the device according to claim 29. Advantageous developments of the method according to the invention and of the device according to the invention are given by the respective dependent claims.

According to the invention, the fluid to be analysed is brought into contact with particles, on the surface of which are disposed first collector substances which are specific for the analyte and which bind the analyte. Suitable as collector substances are for example antibodies, antibody fragments, or in general receptors for analytes acting as ligands.

The particles are then, or simultaneously, brought into contact with at least one electrode, on the surface of which second collector substances which are specific for the analyte are also disposed. The electrode is here located in an electrolytic solution which contains an electrochemically convertible substance, or it is then introduced into such an electrolytic solution. The particles can here be brought into contact with the fluid to be analysed before being attached to the electrode and only then be introduced to the electrode or to the electrolytic solution, or they are applied to the electrode or introduced into the electrolytic solution jointly with the fluid to be analysed. The binding of the analytes to the particles can therefore take place beforehand or directly in the electrolytic solution.

If now a voltage is applied to the electrode, in the case of electrodes to which no particles are bound, an electrochemical conversion of the electrochemically convertible substance takes place. However if portions of the electrode surface or, insofar as it is a matter of a microelectrode, the entire electrode surface are/is blocked by particles bound in a sandwich arrangement, no electrochemical conversion can take place, such that also no current is observed over the electrode.

What is now critical in the method according to the invention is that a continuously increasing and scaleable force is exerted on the particles such that, when a specific strength of the force is exceeded, the particles are removed from the electrode surface. This directly frees the electrode surface such that now an electrochemical conversion can follow. When a specific strength of the force is reached, therefore, the current over the electrode increases in jumps. The force correlated with this discontinuous increase in current provides information not only about the number of the bound particles on the electrode surface but also about the bonding strength, i.e. about the number of bonds between the electrode and particle due to the sandwich arrangement.

The method according to the invention has now several crucial advantages over the prior art. Firstly, the step function of the current can be simply detected, an exact measured value of the current not being relevant since only the strength of the force gives information about the bond. Also a drift of the electrochemical signal does not influence the analysis. This applies also to varying sample matrices, to the temperature or even electrode effects.

The method according to the invention is very sensitive and has a large signal by comparison with the method from prior art, such that detection of individual particles is possible with a good signal-to-noise ratio. Excess particles in the electrolytic solution are of no consequence for the measurement. What is critical is merely the geometrical covering of the electrode by particles bound in a sandwich arrangement. A calibration of the method or of the device is therefore not necessary.

Using the method of the invention, it is firstly possible to determine the number of bound particles per electrode by determining the number and/or size of the steps in the current measured over the electrode. Secondly, the bonding strength is determined by the number of sandwich complexes which in turn is determined by the concentration of analyte. The force which is measured during a discontinuous increase in the current flow, is consequently correlated with the concentration of analyte. This also makes possible a simple calibration of the entire arrangement in advance, insofar as desired.

Advantageously a voltage which has both a proportion of d.c. voltage and a proportion of a.c. voltage can be applied. The a.c. voltage portion is here so set that it alone causes a conversion of the electrochemically convertible substance and thus a flow of current. To determine the step function of the current flow, however, only the electrical current which is caused by the a.c. voltage is detected. By a method management of this type, the discontinuity point, at which the particles are detached from the electrode as a result of the external force, can be detected more exactly.

Particularly advantageous ways of managing the method, which make it possible to determine the forces actually acting on the particles and permit simultaneous measurement and calibration, arise if, when an array comprising many individual electrodes is used, not all the electrodes are uniformly covered with antibodies, but some electrodes have for example, instead of the antibodies, a layer with immobilised biotin. Then particles which do not themselves bear any antibodies can also be bound. Instead of this, the particle surface can also, for example, be covered with avidin. In this case, the particles can be fixed on the electrode via an avidin-biotin bond. This bonding of particles independently of the concentration of analytes can now take place at different strength.

With identical covering of the electrode with biotin, particles which have little avidin on their surface can only be weakly bound, whereas particles, the surface of which has a larger amount of avidin, can form a considerably stronger bond. The amount of avidin on the particle surface can be set by the chemical functionalisation process of the particles.

If now the corresponding particles are added to the electrode array, which can bind both the particles covered with antibodies and the particles covered with two different amounts of avidin, all three types are held on the electrode with different binding forces. When the force acting on the particles is increased, then first the weakly bound particles are removed from the electrode and subsequently those particles having a stronger bond. The points in time at which the particles covered with different amounts of avidin are detached serve as a measurement for the actually effective force on the particles. Differences in magnetic behaviour in the case of different types of particles (e.g. if the sensor is to be operated later with a different batch from the manufacturer, a batch from a different manufacturer or with other magnetic material), can be eliminated from the measurement curve in this way. Since the binding forces for the particles bound with biotin-avidin complexes are known and are easily reproducible, there is thus also the possibility of undertaking in a single working step both a calibration of the measurement system and determining the detachment force of particles which were bound by means of antigen-antibody interactions.

Another method, with which measurement and calibration can take place simultaneously, uses, in addition to the particles covered with antibodies, two further types of particles, of which each type is so covered with a different substance on the surface that the two types of particles are bound to the electrode with a different force and independently of the concentration of analyte.

In the same way, for the calibration, as described in the above example, some of the electrodes of the array can be covered e.g. with biotin and in addition to the particles covered with antibodies, particles can be added which have e.g. a constant avidin covering of the surface. If the particles covered with avidin are now magnetisable to different degrees or have different diameters, through the action of a single force generated from outside two different forces can be exerted on the avidin-biotin bond. Here, too, the reproducible binding force of the avidin-biotin bond can be used in order to determine the force which is actually effective and acting on the particles bound to the remaining electrodes by means of other mechanisms, e.g. antibody-antigen interactions.

In a further method, all the electrodes are uniformly covered with collector molecules. Then simultaneous measurement and calibration can take place if, in addition to the analyte and the particles covered with collector molecules, particles without collector molecules are also added, the surface of which is directly covered with a specific number of analyte molecules. For example, in addition two types of particles are added, the surface of which in the one case already has many analyte molecules and in the other case only a few analyte molecules. After all three types of particles have come into contact with the sample, they bind to the electrodes with differing bonding strength and are then removed from the electrodes again by forces of differing strength. If the concentration of the analyte molecules on the particles without collector molecules is known, the forces which are necessary for removing these particles from the electrode can be used to determine the analyte molecules on the particles with collector molecules and thus to determine the concentration of the analyte.

What is furthermore advantageous is that in the case of the method according to the invention, no separate washing step or cleaning of the sample has to take place, since non-bound excess particles do not have any significance for the method or the measurement. These are even removed from the electrode at the beginning of the measurement by a small force.

What must be remembered is that the method according to the invention can be carried out in a plurality of variant ways. Firstly, the analyte and particle can be pre-incubated and then transferred to a measuring cell with the electrode, or even be mixed in the measuring cell itself. Secondly, the electrochemically convertible substance can either be already contained in the measurement cell, can be added to said cell subsequently, or can even be produced in the measuring cell itself in advance or subsequently.

As the force which is exerted on the particles, mechanical forces can be considered which are generated for example by flow or sound, as can electrical or magnetic forces, insofar as the particles have electrical or magnetic properties. In the latter case it is also possible to focus the particles in advance onto the electrode by means of an electrical or magnetic field and in this way to further increase the sensitivity of the entire arrangement and of the entire method.

Instead of one electrode, a plurality of electrodes can also be used, for example disposed in an electrode array. These can be connected or coupled as a totality or can also be measured connected separately.

If microelectrodes are used as the electrodes, especially those with a size of their surface comparable to the cross-section of the particles, a particularly defined step function is observed, since with a bound particle practically no electrochemical conversion takes place, whilst without bound particles this conversion reaches its maximum value. The electrodes can furthermore be embedded in an insulator matrix in which the properties of each electrode can be influenced specifically.

Magnetic beads having a particle diameter of between 1 μm and 3 μm are used particularly advantageously as particles, the surface of which is modified in order to bind and immobilise the collector substances.

The amperometric measuring method according to the invention will be explained in what follows by way of example.

In a measuring cell are located a working electrode, a counter-electrode and possibly a reference electrode in addition. The working electrode (preferably a microelectrode) is located on a chip, on which the counter and reference electrodes can also be accommodated. The measuring cell is filled with an electrolytic solution which already contains an electrochemically convertible substance. However this substance can also be added subsequently or generated during the analysis process. Furthermore, analyte molecules, suitable collector molecules for the analyte, and microparticles which can also bind the analyte are to be found there.

A constant potential is applied to the working electrode. If this potential is sufficiently large for an electrochemical conversion of the electroactive substance to be possible, an electrical current flows through the solution. This depends, inter alia, on the type of electroactive substance, the electrode surface, the diffusion coefficient and the mass transfer in the solution. Redox reactions and other electrochemical reactions which lead to a current flow are suitable for the electrochemical conversion. In many cases, potentials in the range between −1 V and +1 V are adequate. Individual platinum electrodes having a diameter of 1-5 μm can serve as the electrodes, but larger or even smaller electrodes can also be used. Such microelectrodes can be used individually or also be connected in parallel as an electrode array. The detection of microparticles can take place, alternatively to the described method, also by applying a constant potential which is overlaid with an a.c. component in addition.

With this device it is possible to detect individual microparticles present in the electrolytic solution. The microparticles added from outside typically have a diameter of 1-3 μm, but can also be smaller or larger depending on the application. Ideally, the microparticles are of spherical shape, but other shapes could also be used. Their external chemical composition plays only an insignificant role in the detection of particles, such that microparticles formed from glass, polystyrene (commonly referred to as latex particles) or also other substances can be used. However it is important that the microparticles are magnetisable, such that a force can be exerted on the microparticles via a magnetic field applied from outside. If the particles are not magnetizable, forces could however be exerted on the particles via other techniques (e.g. by flow or by means of ultrasound). All suitable forces must be capable of being set in a reproducible and scaleable manner.

The current flow registered at the microelectrode, depends, as described, inter alia on the amount of electroactive substance which reaches the electrode in a specific interval of time. If now a microparticle is located in the immediate vicinity of the electrode (directly on the electrodes), fewer electroactive molecules reach the electrode surface per time unit. As a consequence of this, a smaller current flow is determined. If subsequently the particle is removed from the electrode by an external force, the current flow increases again. By determining the current flowing it is also possible to decide whether a particle is located on the electrode or not. Detection of a particle takes place here in a very sensitive manner, for an individual particle on a microelectrode can cause a current alteration of up to 100%. Simultaneously, high selectivity is present, since a change of signal only occurs if a particle is in fact directly located on an electrode. Particles which remain in the solution or bind to other sites of the chip do not result in any influencing of the signal and thus in no interference with same.

If microelectrodes and microparticles are in addition so covered with substances (in a uniform or different manner) that when the analyte to be determined is present, a sandwich formation can take place on the microelectrode (platinum electrode—receptor 1—analyte—receptor 2—microparticle) a reduction of the current flow is also registered here when this sandwich formation actually occurs. Depending on the concentration of analyte, more or less force is now necessary in order to reverse these sandwich formations again (according to the concentration of analyte, more or fewer bonds are involved). In the case of microparticles which can be magnetised, a force can be exerted from outside via a magnetic field on the sandwich bonds. If the force is large enough, the bonds on the analyte are released and the particle is removed from the electrode. The time of separation is registered as a signal in the current flow. In the case of a scaleable force it is thus possible to determine the bonding strength of the analyte molecules and thus to undertake determination of the concentration of the analyte in the examined sample. The prerequisite for this is however that the bonding strength of the analyte inside the sandwich is lower than the other bonding strengths inside the complex (particle—substance 2 or respectively platinum electrode—substance 1). This can be adjusted by appropriate selection of the substances used. In conjunction with the described detection method for microparticles, the detection of extremely few analyte molecules is also possible.

If an individual microelectrode or an array with individually addressable microelectrodes are used as the working electrodes, the respective bonding strength can be measured separately at each electrode. When an array with electrodes connected in parallel is used, an average value of all bonding strengths is registered.

In order to be able to exert a magnetic force on each individual particle which is located on a microelectrode, a suitable magnet (permanent magnet, electromagnet) can be brought close to the particles from outside. By varying the distance from the particles, a variation of the force is possible. In the case of an electromagnet, in addition the force can be adjusted by adjusting the current strength inside the magnetic coils. Furthermore, the microelectrodes which are located on a chip can in each case be surrounded by a microcoil. If the entire system (chip with microelectrodes, microcoils, magnetisable particles, electrolytic solution) is located in a constant or variable magnetic field, which is produced by permanent magnets or electromagnets, a scaleable force can be exerted on the magnetisable particles by an adjustable current flow inside the microcoils on the chip.

Furthermore, a force can be exerted on the bound particles by generating a flow of the electrolyte relative to the chip surface (and thus also relative to the particles). For this purpose, the chip is best located in a fluidics system; the flow is generated for example by a pump. By varying the rate of flow, an alteration in the force on the particles is possible. Also by coupling-in sound fluctuations in the electrolytes, for example ultrasound fluctuations, forces can be exerted on the particles which can lead to the breaking of the sandwich complex. In this process, a variation of the effective forces is possible by a variable coupling of the sound producer to the measuring system or by a variation of the sound production. In both cases (detachment via flow or respectively via sound) the microparticles used can be magnetisable; however this is not a necessary prerequisite here for detecting the particles.

Potential analytes or collector substances arise due to their properties such as e.g. the molecular interactions between receptor-ligand, antibody-antigen, antibody-hapten, antibody fragment-antigen, aptamers, proteins, nucleic acids, oligonucleotides, DNA,RNA and interaction with cells.

The method can be described using a protein as an example. For the sake of simplicity, the immobilisation of an anti-mouse antibody on the electrode :surface and the immobilisation of a mouse-antibody on the bead surface is carried out. In a sandwich configuration, both surfaces can also be covered with anti-mouse antibodies, such that the mouse antibodies act as free analytes. In parallel and with the same function as this method, biotin is immobilised on the surface. On the addition of streptavidin, a strong interaction (bond) between receptor and ligand is formed.

Methods of immobilising collector molecules on platinum surfaces are extensively published, for example in the document DE 100 36 907.3 of the present applicant, U.S. Pat. No. 5,436,161 or WO 94/06485. Magnetic beads having a diameter of 0.1-5 μm are preferably used. The magnetic material of the beads is usually encased in a polymer layer (e.g. latex, polyvinyl) and is pre-activated in a standard manner with chemically functional groups. The beads can be covered with antibodies for example in this way using methods such as EDC/NHS activation. Streptavidin beads and also beads with different receptors are commercially available.

The figures show:

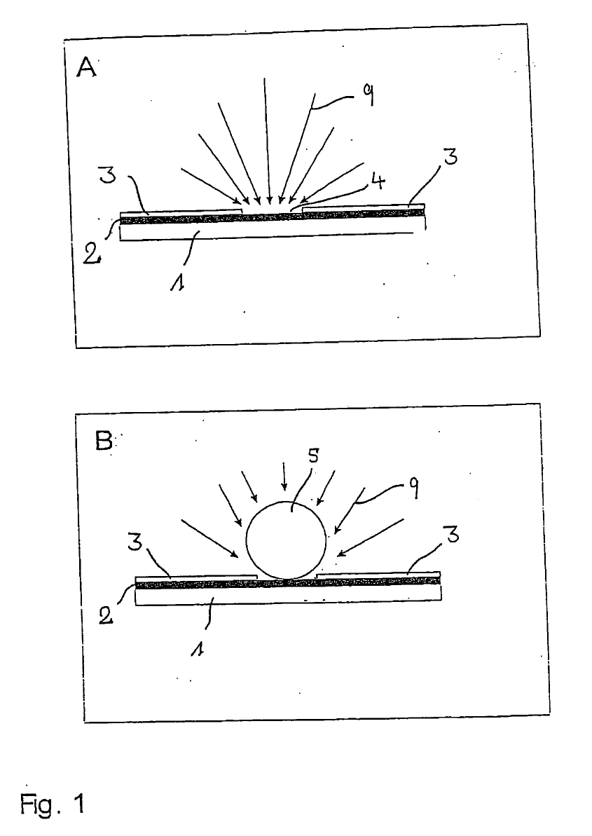

FIG. 1A a detail of a chip with a microelectrode;

FIG. 1B the detail of FIG. 1A with bound microparticle;

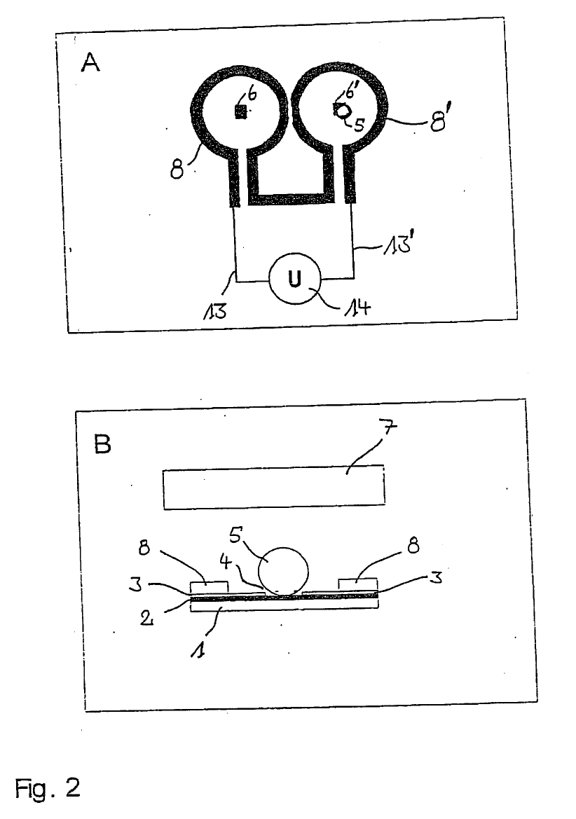

FIG. 2A a possible arrangement of microelectrodes and microcoils according to the invention;

FIG. 2B a further possible arrangement of a microelectrode with a magnet according to the invention;

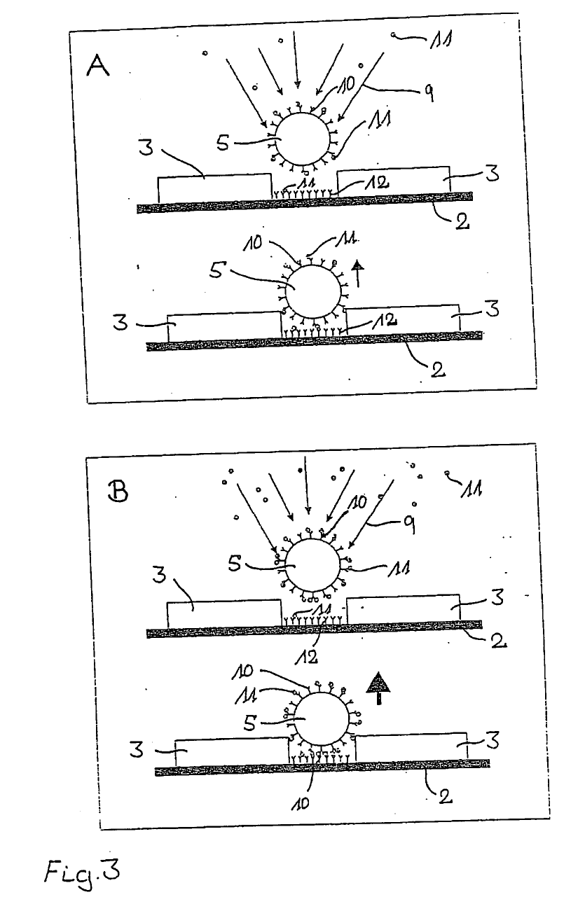

FIG. 3 two examples of the method according to the invention with low and high concentrations of analytes.

FIG. 1A shows the detail of a chip with a microelectrode which can be produced using current thin-film or lithographic methods of the semi-conductor industry. The chip has a support layer 1, to which a metal layer 2 formed from platinum is applied. This metal layer 2 is covered by an insulating layer 3 in such a way that a circular aperture 4 remains in the insulator on which the surface of the platinum layer 2 is exposed. The reference numeral 9 designates the field lines of a diffusion field being set at the electrode 2 by an applied electrical field.

Instead of one electrode, correspondingly also a plurality of electrodes could naturally be present. The diffusion field of the microelectrode which is indicated by the field lines 9, is generally produced during an electrochemical reaction at the electrode after some time.

FIG. 1B shows the same arrangement, however a microparticle 5 is bound to the exposed surface of the metal layer 2. This leads to the electrochemical substance conversion being greatly reduced at the electrode. This leads to an alteration in the diffusion field in front of the microelectrode 2, as is also represented by the field lines 9.

FIG. 2A shows a possible arrangement of two microelectrodes 6, 6′ on a substrate, in plan view. These microelectrodes 6, 6′ are surrounded by conductor tracks 8, 8′ which represent microcoils.

These micro-coils are provided with current via electrical supply lines 13, 13′ from a voltage supply 14. By means of these microcoils, magnetic fields can be exerted on microparticles according to the invention, in order to focus same on the electrodes 6, 6′ and then to exert a continuously rising, scaleable magnetic force on bound microparticles 5 in order to detach same from the electrodes 6, 6′. At the moment of the detachment of the microparticle, there is a discontinuous rise in the electrochemical conversion and thus in the current flow through the electrode, such that the associated force can be registered and detected as a measurement for the number of binding sites and thus also as a measurement for the concentration of analytes.

FIG. 2B shows, on the other hand, a corresponding arrangement in which, in addition to the coil 8 which surrounds the electrode, a further magnet 7, for example a permanent magnet or an electromagnet is so disposed that it exerts a force on the microparticle 5 said force pointing away from the electrode surface. The magnet 7 can be brought close to the electrode or distanced from same, such that the force exerted on the microparticle 5 can be scaled.

FIG. 3 shows again the measuring principle of the present invention, in FIG. 3A the case with a low concentration of analytes and in FIG. 3B a case with a high concentration of analytes being represented. In FIG. 3A the upper diagram shows a microparticle 5, on the surface of which antibodies 10 are immobilised. Only a small portion of these antibodies 10 is taken up with the analyte 11 acting as an antigen. In addition, free antigens 11 are located in the analyte solution.

Furthermore on the surface of the platinum electrode 2 are located antibodies 12, which are also different from antibodies 10 and which also bind antigens 11. In the lower illustration in 3A it can be recognised how the particle 5 is bound via a sandwich arrangement to the electrode surface 12 and the entire free electrode surface is filled and thus any kind of electrochemical conversion on the free electrode surface is prevented. The particle is here bound to the free electrode surface merely by a sandwich arrangement, such that a low force, indicated by the arrow in the figure, is sufficient to detach the particle from the electrode surface. The size of the force consequently provides information about the number of the sandwich arrangements, i.e. the bond of the particles to the free electrode surface and thus about the concentration of the analyte in the solution to be analysed.

FIG. 3A shows the same arrangement with a high concentration of analyte in the solution to be analysed. What can be recognised is that most of the antibodies 10 immobilised on the particle are occupied with the analyte-antigen 11. In the lower illustration in 3B it can be recognised that altogether four sandwich arrangements are formed. The force represented by the arrow, which is necessary to detach the particle 5 from the free electrode surface is consequently considerably greater than in the case of the low concentration of analyte from FIG. 3A. Here, too, consequently the necessary force for detaching the particle 5 from the free electrode surface provides a measurement for the concentration of the analyte in the solution to be analysed.

1. Method of detecting and/or determining the concentration of analytes in a fluid to be analysed,
characterised in that,
the fluid to be analysed is brought into contact with particles, on the surface of which first collector substances, which are specific for the analyte and which bind the analyte, are disposed, the particles are then, or simultaneously, brought into contact with at least one electrode, on the surface of which second collector substances which are specific for the analyte and which bind the analyte, are disposed, and which is located in an electrolytic solution which contains an electrochemically convertible substance, or is then introduced into same,
a voltage is applied to the electrode and a force directed away from the electrode surface is exerted at variable strength on the particles and the force is detected at which the current flow over the electrode increases.

2. Method according to the preceding claim, characterised in that a voltage having a proportion of d.c. voltage, which produces a conversion of the electrochemically convertible substance, and a proportion of a.c. voltage is applied to the electrode.

3. Method according to the preceding claim, characterised in that the current flow produced in the electrolytic solution by the a.c. voltage portion is detected.

4. Method according to one of the preceding claims, characterised in that particles having magnetic and/or electrical properties are used.

5. Method according to the preceding claim, characterised in that electrically charged or electrically polarisable particles are used.

6. Method according to one of the two preceding claims, characterised in that particles are used which have at least partially paramagnetic, diamagnetic, ferromagnetic, ferrimagnetic or antiferromagnetic properties.

7. Method according to one of the preceding claims, characterised in that a mechanical, electrical and/or magnetic force is exerted on the particles which is directed away from the electrode surface.

8. Method according to the preceding claim, characterised in that a force is exerted on the particles by means of a homogenous or inhomogeneous, magnetic and/or electrical field, by means of a flow, by means of sound, ultrasound and/or by means of a temperature increase.

9. Method according to one of the two preceding claims, characterised in that the force exerted on the particles increases continuously.

10. Method according to one of the three preceding claims, characterised in that the force is determined at which the current flow through the electrode greatly increases.

11. Method according to one of the preceding claims, characterised in that the particles are first brought into contact with the liquid to be analysed and are then introduced into the electrolytic solution.

12. Method according to one of claims 1 to 11, characterised in that the particles in the electrolytic solution are brought into contact with the analytes.

13. Method according to one of the preceding claims, characterized in that the particles are transported (focused) to the electrode before the exertion of the mentioned force.

14. Method according to the preceding claim, characterized in that the particles are transported to the electrode by means of sedimentation, an electrical field and/or a magnetic field.

15. Method according to one of the preceding claims, characterized in that the first and second collector substances are identical substances.

16. Method according to one of claims 1 to 15, characterised in that the first and second collector substances bind different regions of the analyte.

17. Method according to one of the preceding claims, characterised in that the electrochemically convertible substance in the electrolytic solution is produced before or after the particles have been brought into contact with the electrode in the electrolytic solution, or are added to said solution.

18. Method according to one of the preceding claims, characterised in that an array of electrodes is used as the electrodes.

19. Method according to one of the preceding claims, characterised in that microelectrodes are used as the electrodes.

20. Method according to one of the preceding claims, characterised in that an electrode is used which has a size of surface comparable to the cross-section of the particles.

21. Method according to the preceding claim, characterized in that microelectrodes having a diameter of between 1 μm and 5 μm are used as the electrodes.

22. Method according to one of the preceding claims, characterised in that electrodes formed from platinum, gold or carbon are used.

23. Method according to one of the preceding claims, characterised in that electrodes are used which are embedded in an insulator matrix.

24. Method according to one of the preceding claims, characterized in that collector molecules are used which interact with the analyte via receptor-ligand interaction, antibody-antigen interaction, antibody-hapten interaction, antibody-antigen interaction or the like.

25. Method according to one of the preceding claims, characterised in that aptamers, proteins, nucleic acids, oligonucleotides, DNA, RNA and/or entire cells or cell fragments are used as the collector substances.

26. Method according to one of the preceding claims, characterised in that particles which contain glass or polystyrene are used.

27. Method according to one of the preceding claims, characterised in that beads, especially magnetic beads, are used as the particles, on the surface of which collector substances, especially collector molecules, are immobilised.

28. Method according to one of the preceding claims, characterised in that particles having a diameter of between 1 μm and 5 μm are used.

29. Device for detecting and/or determining the concentration of analytes in a fluid to be analysed, using a method according to one of the preceding claims,
characterised by
at least one electrode, on the surface of which collector substances which are specific for the analyte and which bind the analyte are disposed,
a device for applying an electrical voltage to the electrode which causes an electrochemical conversion of a substance in an electrolytic solution in which the electrode is located,
a device for generating a scaleable force on particles which are bound to the surface of the electrode, as well as
a device for detecting the change in the current flow over the electrode, whilst a force is exerted by the device for generating a scaleable force in such a way that particles bound to the electrode are detached from the electrode.

30. Device according to the preceding claim, characterised by a plurality of electrodes, on the surface of which collector substances which are specific for the analyte and which bind the analyte are disposed.

31. Device according to one of the two preceding claims, characterized in that the collector substances contain receptors for the analytes or antibodies or respectively antibody fragments, which are directed against the analytes.

32. Device according to one of the three preceding claims, characterised in that the electrode or electrodes are microelectrodes.

33. Device according to the preceding claim, characterised in that the microelectrodes have a diameter of between 1 μm and 5 μm.

34. Device according to one of claims 29 to 33, characterised in that the electrode or electrodes is/are disposed on a substrate.

35. Device according to one of claims 29 to 34, characterised in that the electrodes are incorporated in an inert matrix.

36. Device according to one of claims 29 to 35, characterised in that it has at least one magnet, by means of which a magnetic force can be exerted on the substances disposed on the surface of the electrode or electrodes.

37. Device according to the preceding claim, characterised in that the magnet is a permanent magnet and/or an electromagnet.

38. Device according to one of the two preceding claims, characterised in that the spacing between the electrode or the electrodes and the magnet is variable.

39. Device according to one of claims 29 to 38, characterised in that at least one of the electrodes is surrounded by a coil.

40. Device according to one of claims 29 to 39, characterised in that the collector substances are aptamers, proteins, nucleic acids, oligonucleotides, DNA, RNA and/or entire cells or cell fragments.

41. Use of a method and/or a device according to one of the preceding claims to detect aptamers, proteins, nucleic acids, oligonucleotides, DNA, RNA and/or entire cells or cell fragments as analytes.
